(19) United States
(12) Patent Application Publication
(10) Pub. No.: US 2008/0138414 A1
(43) Pub. Date: Jun. 12, 2008

(54) Methods of Regenerating Cartilage

(75) Inventors: James Huckle, Northallerton (GB); Sarah Freestone, South Milford (GB); Kelly Langford, York (GB); Drew Burdon, Harrogate (GB)

Correspondence Address: NORMAN F. HAINER, JR.;SMITH & NEPHEW, INC., 150 MINUTEMAN ROAD, ANDOVER, MA, 01801, US

(73) Assignee: Smith & Nephew, Inc., Andover, MA (US)

(21) Appl. No.: 11/951,676

(22) Filed: Dec. 6, 2007

Related U.S. Application Data
(60) Provisional application No. 60/869,123, filed on Dec. 8, 2006.

(51) Int. Cl. A61K 35/00; A61P 19/00; A61K 35/28; A61K 35/32

(52) U.S. Cl. 424/484; 424/93.7; 424/577; 424/548

(57) ABSTRACT

The present disclosure relates to methods of regenerating cartilage. In an embodiment, a method includes initiating a release of precursor cells, including bone marrow cells and progenitor cells, into a cartilage defect; and applying a population of exogenous cells to the cartilage defect. The exogenous cells, selected from a group including chondrocytes, synoviocytes, fat pad cells, chondroprogenitor cells, mesenchymal stem cells, and any combination thereof, induce the precursor cells to form cartilage tissue through a release of factors by the exogenous cells. The factors stimulate the precursor cells to form cartilage cells. The cartilage cells then form cartilage tissue. The factors are selected from a group including transforming growth factors, fibroblast growth factors, platelet-derived growth factors, insulin-like growth factors, epidermal growth factors, interleukins, and any combination thereof. Other methods of regenerating cartilage are also disclosed.

### CROSS REFERENCE TO RELATED APPLICATIONS

This application claims the benefit of U.S. Provisional Application No. 60/869,123, filed Dec. 8, 2006, the disclosure of which is incorporated herein by reference in its entirety.

### BACKGROUND OF THE INVENTION

1. Field of the Invention

The present disclosure relates to the treatment and repair of defects or lesions in cartilage. More specifically, the disclosure relates to methods of regenerating cartilage to form a more hyaline-like repair of a cartilage defect or lesion.

2. Related Art

Articular cartilage defects within the knee are frequently observed in a broad spectrum of patients. These articular cartilage lesions are difficult to repair and as such represent a major challenge to surgeons. Moreover, the current treatments available today yield a fibrous repair wherein a fibrocartilage is formed that provides only temporary relief The fibrocartilage tissue does not have the same mechanical properties as the natural hyaline cartilage found in the joint surfaces and degrades faster over time as a consequence of wear. Patients typically have to undergo repeated surgical procedures to relieve reoccurring symptoms, though this type of surgery does not delay or prevent further deterioration of the cartilage surface.

Current treatments include synthetic, bioabsorbable implant structures that are inserted into the defect and that contain pores and/or channels into which the surrounding tissue grows as the structure erodes, thus providing new tissue growth of roughly the same size and shape as the implant. In addition to creating a fibrocartilage type tissue and therefore only providing temporary relief, this may require the creation of a large osteochondral defect within which the implant is inserted. The creation of this large defect causes damage to the underlying subchondral bone.

More recently, experimental approaches involving the implantation of autologous chondrocytes have been used in cartilage repair. The process involves the harvest of a small biopsy of articular cartilage in a first surgical procedure, which is then transported to a laboratory specialized in cell culture for amplification. The tissue biopsy is treated with enzymes that will release the chondrocytes from the matrix, and the isolated cells will be grown for a period of 3 to 4 weeks using standard tissue culture techniques. Once the cell population has reached a target number, the cells are sent back to the surgeon for implantation during a second surgical procedure. This manual labor-intense process is extremely costly and time consuming. In addition to the prohibitive cost, there is a traumatic impact to the patient of having two surgical procedures to the knee. Furthermore, the quality of the autologous cells and their minimum expansion capability limits the benefits of performing this procedure.

In addition to the above approaches, a surgeon will use a variety of methods such as abrading, drilling, or creating microfractures in the bone below the defect, otherwise known as subchondral bone, to induce bleeding into the defect and allow the formation of a clot. The cells coming from the bone marrow form a scar-like tissue called fibrocartilage, as mentioned above, that provides only temporary relief. Recently, microfracture has been used in conjunction with the bioabsorbable implant structures mentioned above to allow cells from the bone marrow to populate the material and form a matrice within the defect. In addition, the tissue surrounding the structure grows into the pores as the material erodes, thus providing new tissue growth of roughly the same size and shape as the substrate. Unfortunately, fibrocartilage tissue is also formed by the use of this approach.

A more recent approach has been to load tissue cells, such as the above mentioned autologous chondrocytes and undifferentiated mesenchymal stem cells, onto the bioabsorbable substrate mentioned above before implantation of the substrate into the defect. This procedure is not used in conjunction with a microfracture procedure. It is believed that placing these cells into an environment favorable to cartilage formation will allow the cells to differentiate into chondrocytes. However, these cells do not differentiate into chondrocytes and only promote poor quality fibrous cartilage repair. Integration of cells on the substrate with the existing cartilage does not occur.

Therefore, methods of regenerating cartilage to form a more hyaline-like repair of a cartilage defect or lesion is needed. These methods would provide the defect with cartilage that has mechanical properties and lasting times that are similar to hyaline cartilage. This would substantially reduce any further deterioration of the cartilage surface and consequently any further surgeries to the patient.

### SUMMARY OF THE INVENTION

In one aspect, the present disclosure relates to a method of regenerating cartilage. The method includes initiating a release of precursor cells into a cartilage defect; and applying a population of exogenous cells to the cartilage defect. The exogenous cells induce the precursor cells to form cartilage tissue. Initiating the release of precursor cells into a cartilage defect includes preparing a subchondral bone surface for the release of the precursor cells. In an embodiment, the exogenous cells are associated with a natural or bioabsorbable synthetic material. In another embodiment, the exogenous cells are located within or on a surface of the natural or bioabsorbable synthetic material. In yet another embodiment, the method further includes placing a matrix material adjacent to the natural or bioabsorbable synthetic material. In a further embodiment, the matrix material includes a bioabsorbable porous material. In yet a further embodiment, the natural or bioabsorbable synthetic material surrounds a matrix material.

In an embodiment, the precursor cells include bone marrow cells and progenitor cells. In another embodiment, the progenitor cells are selected from a group including adipoprogenitor cells, osteoprogenitor cells, chondroprogenitor cells, hemapoeitic cells, and any combinations thereof. In yet another embodiment, the exogenous cells are selected from a group including chondrocytes, synoviocytes, fat pad cells, mesenchymal stem cells, chondroprogenitor cells, and any combination thereof. In a further embodiment, the mesenchymal stem cells are selected from a group including differentiated, undifferentiated, allogenic, autologous, and any combination thereof. In yet a further embodiment, the cartilage defect includes a full thickness or partial thickness defect. In an embodiment, the exogenous cells induce the precursor cells to form cartilage tissue through a release of factors by the exogenous cells, the factors stimulating the precursor cells to form cartilage cells, the cartilage cells forming cartilage tissue. In another embodiment, the factors are selected from a group including transforming growth factors, fibroblast growth factors, platelet-derived growth factors, insulin-like growth factors, epidermal growth factors, interleukins, and any combinations thereof

In another aspect, the present disclosure relates to a method of regenerating cartilage including initiating a release of precursor cells into a cartilage defect, in which the precursor cells form a cell clot in the defect; and securing a population of exogenous cells over a top of the defect. The exogenous cells induce the precursor cells in the cell clot to form cartilage tissue. Initiating the release of precursor cells into a cartilage defect includes preparing a subchondral bone surface for the release of the precursor cells. In an embodiment, the exogenous cells are associated with a natural or bioabsorbable synthetic material. In another embodiment, the exogenous cells are located within or on a surface of the natural or bioabsorbable synthetic material. In yet another embodiment, the method further includes placing a matrix material between the natural or bioabsorbable synthetic material. In a further embodiment, the matrix material includes a bioabsorbable porous material.

In an embodiment, the precursor cells include bone marrow cells and progenitor cells. In another embodiment, the progenitor cells are selected from a group including adipoprogenitor cells, osteoprogenitor cells, chondroprogenitor cells, hemapoeitic cells, and any combinations thereof In yet another embodiment, the exogenous cells are selected from a group including chondrocytes, synoviocytes, fat pad cells, mesenchymal stem cells, chondroprogenitor cells, and any combination thereof. In a further embodiment, the mesenchymal stem cells are selected from a group including differentiated, undifferentiated, allogenic, autologous, and any combination thereof In yet a further embodiment, the cartilage defect includes a full thickness or partial thickness defect. In an embodiment, the exogenous cells induce the precursor cells to form cartilage tissue through a release of factors by the exogenous cells, the factors stimulating the precursor cells to form cartilage cells, the cartilage cells forming cartilage tissue. In another embodiment, the factors are selected from a group including transforming growth factors, fibroblast growth factors, platelet-derived growth factors, insulin-like growth factors, epidermal growth factors, interleukins, and any combinations thereof.

In yet another aspect, the present disclosure relates to a method of regenerating cartilage including initiating a release of precursor cells into a cartilage defect; and placing a tissue paste in the defect, wherein the tissue paste includes tissue selected from a group including bone, cartilage, synovium, fat pad, and any combination thereof. The precursor cells induce the tissue to form cartilage tissue. In an embodiment, initiating the release of precursor cells into a cartilage defect includes preparing a subchondral bone surface for the release of the precursor cells. In another embodiment, the method further includes injecting exogenous cells into the defect, wherein the exogenous cells induce the tissue to form cartilage tissue. In yet another embodiment, the method further includes securing a population of exogenous cells over a top of the defect, the population of exogenous cells inducing the tissue to form cartilage tissue. In a further embodiment, the population of exogenous cells is associated with a natural or bioabsorbable synthetic material. In yet a further embodiment, the population of exogenous cells is located within or on the natural or bioabsorbable synthetic material.

In yet a further embodiment, the exogenous cells are selected from a group including chondrocytes, synoviocytes, fat pad cells, mesenchymal stem cells, chondroprogenitor cells, and any combination thereof. In an embodiment, the mesenchymal stem cells are selected from a group including differentiated, undifferentiated, allogenic, autologous, and any combination thereof. In another embodiment, the cartilage defect includes a full thickness or partial thickness defect. In yet another embodiment, the precursor cells or exogenous cells induce the tissue to form cartilage tissue through a release of factors by the precursor or exogenous cells, the factors stimulating the tissue to form cartilage tissue. In a further embodiment, the tissue is in a bioabsorbable carrier. In yet a further embodiment, the exogenous cells are in a bioabsorbable carrier.

In a further aspect, the present disclosure relates to a method of regenerating cartilage including initiating a release of precursor cells into a cartilage defect, in which the precursor cells then form a cell clot in the defect; and injecting the cartilage defect with exogenous cells, wherein the exogenous cells induce the precursor cells in the cell clot to form cartilage tissue. In an embodiment, initiating the release of precursor cells into a cartilage defect includes preparing a subchondral bone surface for the release of the precursor cells. In another embodiment, the method further includes placing a matrix material between the exogenous cells and the defect. In yet another embodiment, the precursor cells include bone marrow cells and progenitor cells. In a further embodiment, the progenitor cells are selected from a group including adipoprogenitor cells, osteoprogenitor cells, chondroprogenitor cells, hemapocitic cells, and any combination thereof. In yet a further embodiment, the exogenous cells are selected from a group including chondrocytes, synoviocytes, fat pad cells, mesenchymal stem cells, chodroprogenitor cells, and any combination thereof.

In an embodiment, the mesenchymal stem cells are selected from a group including differentiated, undifferentiated, allogenic, autologous, and any combination thereof. In another embodiment, the cartilage defect includes a full thickness or partial thickness defect. In yet another embodiment, the exogenous cells induce the precursor cells in the cell clot to form cartilage tissue through a release of factors by the exogenous cells, the factors stimulating the precursor cells to form cartilage cells, the cartilage cells forming cartilage tissue. In a further embodiment, the factors are selected from a group consisting essentially of transforming growth factors, fibroblast growth factors, platelet-derived growth factors, insulin-like growth factors, epidermal growth factors, interleukins, and any combination thereof. In yet a further embodiment, the exogenous cells are in a bioabsorbable carrier.

In yet a further aspect, the present disclosure relates to a method of regenerating cartilage including creating at least one hole in a bone lying below a cartilage defect; placing a matrix material into the hole; and securing a population of exogenous cells over a top of the defect, wherein the exogenous cells induce the precursor cells to form cartilage tissue. In an embodiment, the precursor cells infiltrate the matrix material to form a cell clot. In another embodiment, the exogenous cells are associated with a natural or bioabsorbable synthetic material. In yet another embodiment, the exogenous cells are located within or on a surface of the natural or bioabsorbable synthetic material. In a further embodiment, the matrix material includes a bioabsorbable porous material.

In an embodiment, the precursor cells include bone marrow cells and progenitor cells. In another embodiment, the progenitor cells are selected from a group including adipoprogenitor cells, osteoprogenitor cells, chondroprogenitor cells, hemapoeitic cells, and any combination thereof In yet another embodiment, the exogenous cells are selected from a group including chondrocytes, synoviocytes, fat pad cells, chondroprogenitor cells, mesenchymal stem cells, and any combination thereof In a further embodiment, the cartilage defect includes a full thickness or partial thickness defect. In yet a further embodiment, the exogenous cells induce the precursor cells to form cartilage tissue through a release of factors by the exogenous cells, the factors stimulating the precursor cells to form cartilage cells, the cartilage cells forming cartilage tissue. In an embodiment, the factors are selected from a group including transforming growth factors, fibroblast growth factors, platelet-derived growth factors, insulin-like growth factors, epidermal growth factors, interleukins, and any combinations thereof.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### BRIEF DESCRIPTION OF THE DRAWINGS

The accompanying drawings, which are incorporated in and form a part of the specification, illustrate the embodiments of the present disclosure and together with the written description serve to explain the principles, characteristics, and features of the disclosure. In the drawings:

FIG. 1 illustrates a first method of regenerating cartilage.

FIG. 2 illustrates an embodiment of a cartilage defect repair of the first method.

FIG. 3 illustrates an alternative embodiment of a cartilage defect repair of the first method.

FIG. 4 illustrates another alternative embodiment a cartilage defect repair of the first method.

FIG. 5 illustrates yet another alternative embodiment of a cartilage defect repair of the first method.

FIG. 6 illustrates a second method of regenerating cartilage.

FIG. 7 illustrates an embodiment of a cartilage defect repair of the second method.

FIG. 8 illustrates an alternative embodiment of a cartilage defect repair of the second method.

FIG. 9 illustrates a third method of regenerating cartilage.

FIG. 10 illustrates an embodiment of a cartilage defect repair of the third method.

FIG. 11 illustrates another embodiment of a cartilage defect repair of the third method.

FIG. 12 illustrates yet another embodiment of a cartilage defect repair of the third method.

FIG. 13 illustrates a fourth method of regenerating cartilage.

FIG. 14 illustrates an embodiment of a cartilage defect repair of the fourth method.

FIG. 15 illustrates another embodiment of a cartilage defect repair of the fourth method.

FIG. 16 illustrates a fifth method of regenerating cartilage.

FIG. 17 illustrates an embodiment of a cartilage defect repair of the fifth method.

FIGS. 18A-18C show computer-generated illustrations of a cartilage defect after repair using a method of the present disclosure.

FIGS. 19A-19C show computer-generated illustrations of a cartilage defect after repair with a control method.

### DETAILED DESCRIPTION OF THE EMBODIMENTS

The following description of the preferred embodiment(s) is merely exemplary in nature and is in no way intended to limit the disclosure, its application, or uses.

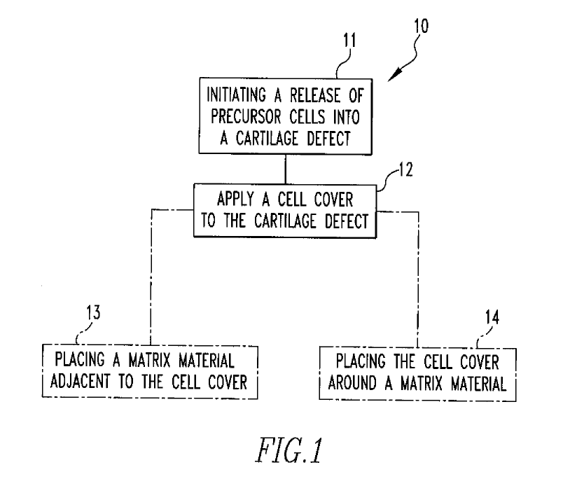

FIG. 1 shows a first method of regenerating cartilage 10. The first method includes initiating a release of precursor cells into a cartilage defect 11. Initiating the release of precursor cells into a cartilage defect includes exposing a subchondral bone surface and creating holes in the subchondral bone surface to initiate the release of the precursor cells. An approach known to those skilled in the art include a microfracture procedure whereby tiny openings are made in the bone lying below the cartilage tissue via the use of microfracture picks and other tools that would create these openings. Other approaches include drilling or scraping through the cartilage into the subchondral bone. The cartilage defect may be a full thickness defect or a partial thickness defect. Full thickness defects are those that penetrate into or through the subchondral bone plate. Partial thickness defects in cartilage tissue are restricted to the cartilage tissue itself. The precursor cells include bone marrow cells and progenitor cells. Progenitor cells are selected from a group including adipoprogenitor cells, osteoprogenitor cells, chondroprogenitor cells, hemapoeitic cells, and any combination thereof

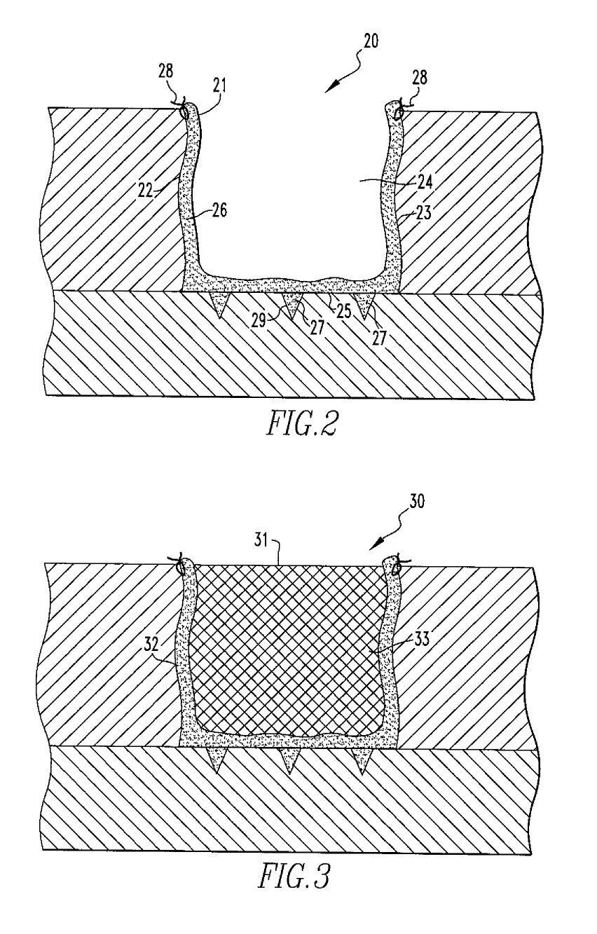

A population of exogenous cells is then applied to the cartilage defect 12. For the purposes of this disclosure, the population of exogenous cells is associated with a natural or bioabsorbable synthetic material, such that the cells are located within or on a surface the material. The natural material may include collagen, fibrin, gelatine, and any other non-synthetic material. The bioabsorbable synthetic material may include a cell cover, cell-seeded felt, cell-seeded film, cell-seeded mesh, cell-seeded gel, or any other synthetic material having cells located within or on a surface of the material. The cells are selected from a group that includes chondrocytes, synoviocytes, fat pad, mesenchymal stem cells, chondroprogenitor cells, and any combination thereof. However, other exogenous cells may be used. The mesenchymal stem cells are selected from a group including differentiated cells, undifferentiated cells, allogenic cells, autologous cells, and any combination thereof An example 20 of a cartilage defect having a cell associated material, such as a cell cover, is shown in FIG. 2. The cell cover 21 is coupled to both sides 22,23 of the defect 24 and lays along the sides 22,23 and the prepared bottom surface 25 of the defect 24.

For the purposes of this disclosure, a prepared bottom surface or prepared defect means that a release of precursor cells into the defect has been initiated via microfracture, shedding, drilling, or any other procedure used to release precursor cells into the defect. The defect in FIG. 2 shows these microfractures 29. The cell cover 21 may be coupled to the sides 22,23 of the defect 24 via several methods 28 including sutures, pins, staples, or an adhesive. Other methods known to those skilled in the art may also be used. The exogenous cells 26 in the cell cover 21 induce the precursor cells 27 to form cartilage tissue through a release of factors that stimulate the precursor cells 27 to form cartilage cells. The cartilage cells then multiply and form cartilage tissue. The factors are selected from a group including transforming growth factors, fibroblast growth factors, platelet-derived growth factors, insulin-like growth factors, epidermal growth factors, interleukins, and any combination thereof. It is believed that the exogenous cells in the cell cover will induce or react with the precursor cells to form a more hyaline-like cartilage, rather than the fibrous cartilage that is formed with microfracture alone.

The method 10 in FIG. 1 may further include placing a matrix material adjacent to the cell cover 13. FIG. 3 shows an example 30 of a matrix material 31 laying adjacent to a cell cover 32 in a prepared defect 33. The matrix material 31 is a biodegradable, porous material as mentioned above. However, as used herein, the matrix material could be any porous composite, solid, or semi-solid substance having pores or spaces sufficiently large to allow cells to populate the substance. The term matrix includes matrix-forming materials, such as those materials that can form matrices within a defect site in cartilage or bone. It is believed that the exogenous cells in the cell cover and the precursor cells will infiltrate the matrix material to form a matrice in the defect site and the exogenous cells will induce the precursor cells to form cartilage tissue via the above-stated method.

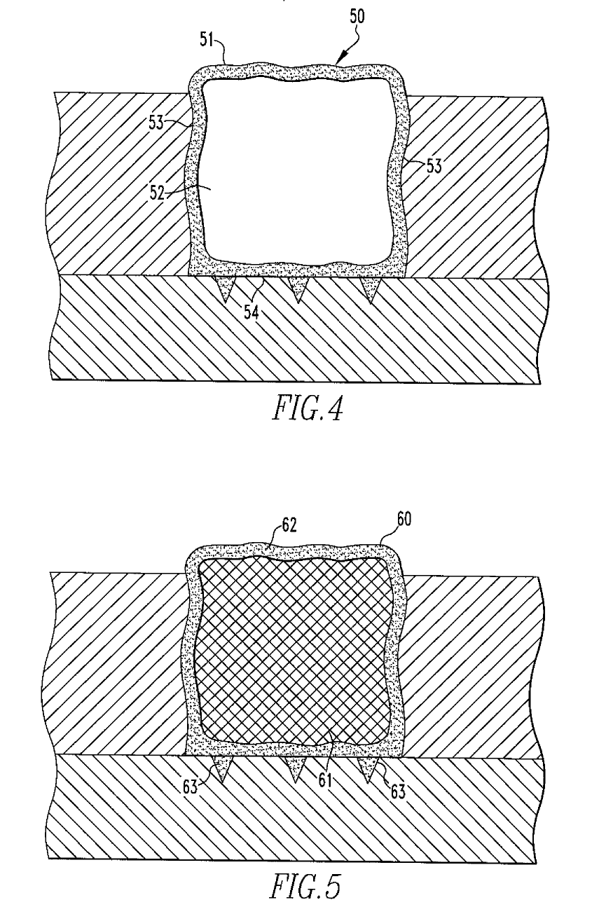

FIG. 4 shows an example 50 of a cell cover 51 in the form of a cell shell that is applied to the cartilage defect 52. Similar to the cell cover in FIGS. 2 and 3, the cell cover 51 lays along the sides 53 and the prepared bottom surface 54 of the defect 52. The method in FIG. 1 may further include placing the cell cover around a matrix material 14. This embodiment is illustrated in FIG. 5, wherein the cell shell 60 surrounds the matrix material 61. As stated above, it is believed that the exogenous cells 62 in the cell shell 60 and the precursor cells 63 will infiltrate the matrix material 61 to form a hyaline-like cartilage tissue matrix through the release of the above-mentioned factors by the exogenous cells in the cell shell 60.

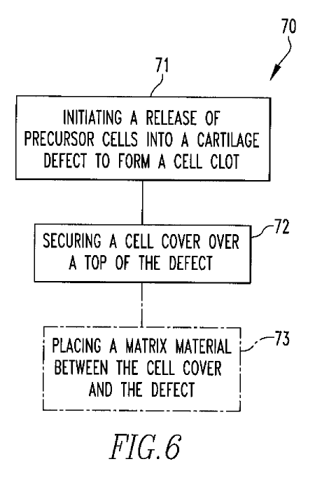

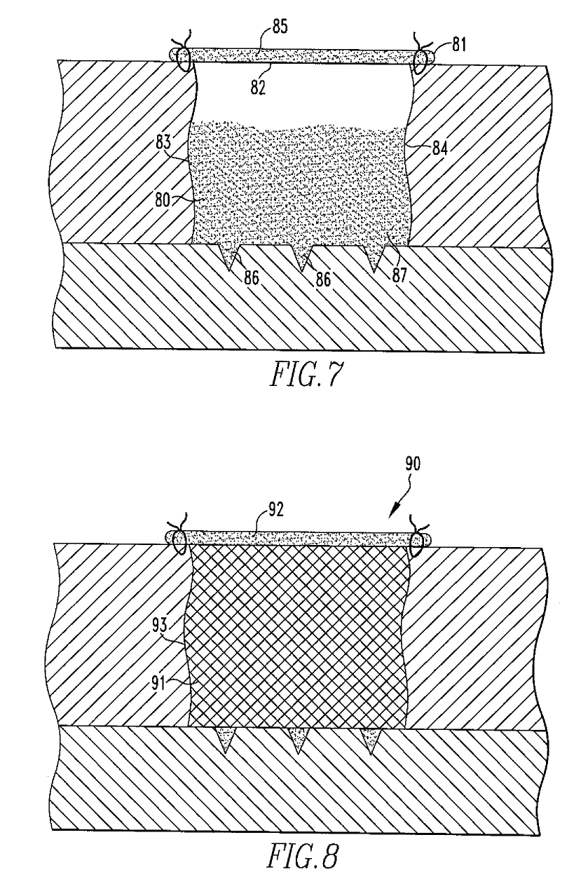

FIG. 6 represents a second method of regenerating cartilage 70. The method includes initiating a release of precursor cells into a cartilage defect wherein the precursor cells form a cell clot or scab in the defect 71. A population of exogenous cells located within or on a surface of a cell cover 81, as discussed above, is then secured over a top of the defect 72. FIG. 7 shows the prepared cartilage defect 80 and the cell cover 81 that is secured over a top 82 of the defect 80. The cell cover 81 may be coupled to the sides 83, 84 of the defect 80 via several methods including sutures, pins, staples, or an adhesive. Other methods of coupling known to those skilled in the art may also be used. Similar to the above method, the exogenous cells 85 in the cell cover 81 induce the precursor cells 86 in the cell clot 87 to form cartilage tissue via the release of the above-mentioned factors by the exogenous cells 85 in the cell cover 81. The method 70 may further include placing a matrix material between the cell cover and the defect 73. An example 90 of this is shown in FIG. 8 wherein the matrix material 91 is placed between the cell cover 92 and the prepared defect 93.

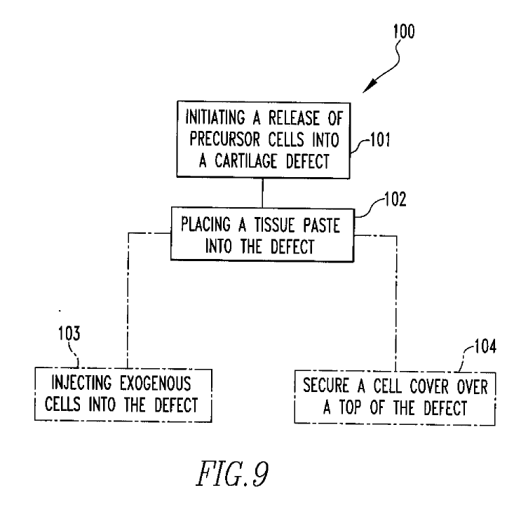

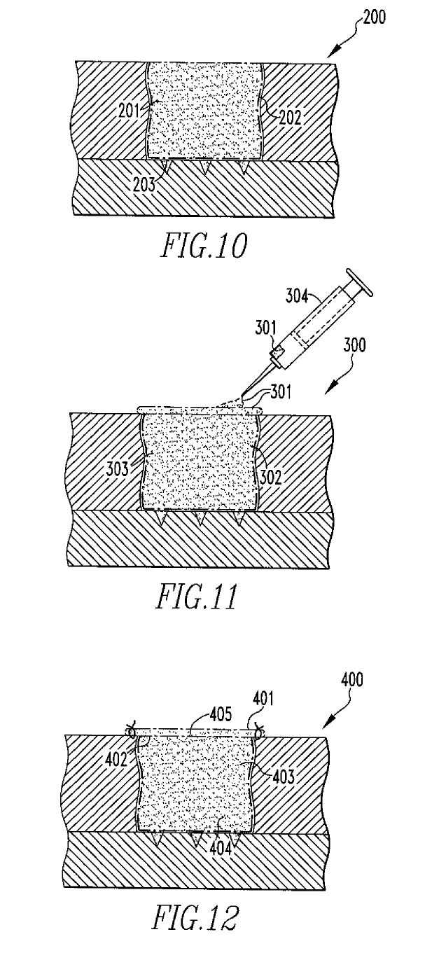

FIG. 9 shows a third method of regenerating cartilage 100. There is an initiated release of precursor cells into a cartilage defect 101 and a tissue paste is then placed adjacent to a bottom surface of the defect 102, wherein tissue within the tissue paste induce the precursor cells to form a cartilage tissue. The tissue paste, which includes minced pieces of tissue that are around 3 mm³ in length, are selected from a group including bone, cartilage, synovium, fat pad, and any combination thereof The minced pieces of tissue are in a bioabsorbable carrier. For the purposes of this disclosure, the bioabsorbable carrier is hyaluronic acid. However, the bioabsorbable carrier may also include derivatives of hyaluronic acid, gelatin, collagen, chitosan, alginate, buffered PBS, Dextran, and any other bioabsorbable carrier known to one of ordinary skill in the art. The paste may be placed in the defect by injection or any other method of placing the tissue paste into the defect. FIG. 10 shows an example 200 wherein the tissue paste 201 sits within the prepared defect 202 and adjacent to the prepared bottom surface 203. The method 100 may further include injecting exogenous cells into the defect 103. An example 300 is shown in FIG. 11 wherein the exogenous cells 301 are injected into the defect 302 after the tissue paste 303 is placed in the defect 302. The exogenous cells 301 may be injected via a syringe 304, as shown in FIG. 11, or via another method known to those skilled in the art. In addition, the cells 301 are in a bioabsorbable carrier. The carrier is similar to the above-mentioned bioabsorbable carriers or other bioabsorbable carriers known to one of skill in the art. The exogenous cells 301 induce the tissue within the paste 303 to form cartilage tissue via the release of any of the above-mentioned factors. The method 100 may alternatively include securing a population of exogenous cells, located within or on a surface of a cell cover, over a top of the defect 104. FIG. 12 shows an example 400 whereby a cell cover 401 is secured to the top 402 of the defect 403. The cell cover 401 is secured to the top 402 of the defect 403 after the tissue paste 404 is placed in the defect 403. Similar to the exogenous cells 301, the exogenous cells 405 in the cell cover 401 induce the tissue paste 404 to form cartilage tissue via the release of any of the above-mentioned factors by the exogenous cells 405 in the cell cover 401.

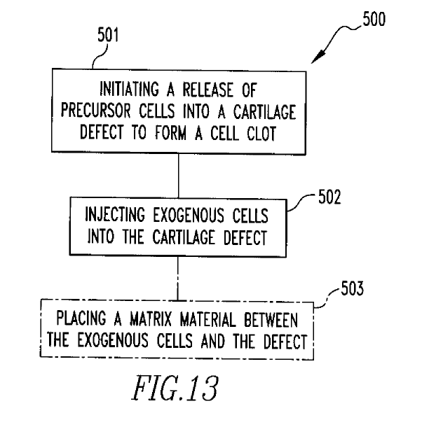

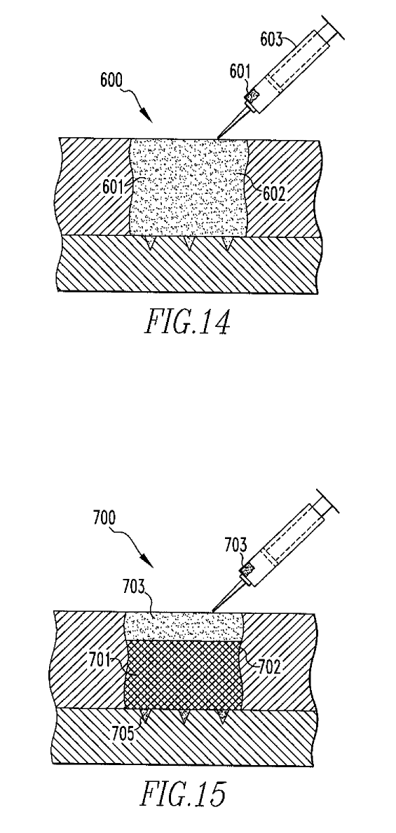

FIG. 13 shows a fourth method 500 of regenerating cartilage including initiating a release of precursor cells into a cartilage defect to form a cell clot 501 and injecting the cartilage defect with exogenous cells 502. The exogenous cells induce the precursor cells in the cell clot to form cartilage tissue. An example 600 is shown in FIG. 14 whereby the exogenous cells 601 are injected into the prepared defect 602 via the use of a syringe 603. Another method known to those of ordinary skill in the art, of placing the exogenous cells 601 into the prepared defect 602, may be used. The method 500 may further include placing a matrix material between the exogenous cells and the defect 503. An example 700 is shown in FIG. 15 wherein the matrix material 701 is placed in the prepared defect 702 and the exogenous cells 703 are then injected into the defect 702. It is believed that the exogenous cells 703 and the precursor cells 705 will infiltrate the matrix material 701 to form a hyaline-like cartilage matrice and the exogenous cells 703 will induce the precursor cells 705 to form cartilage tissue via the above-stated method. Similar to the exogenous cells 301, the exogenous cells 502 are also in a bioabsorbable carrier.

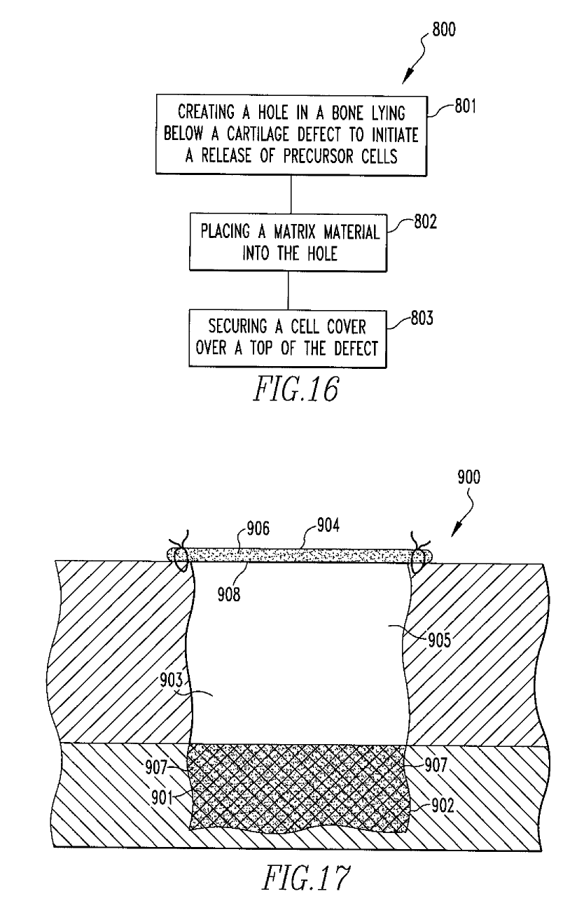

FIG. 16 shows a fifth method 800 of regenerating cartilage, which includes creating at least one hole in a bone lying below a cartilage defect 801, placing a matrix material into the at least one hole 802, and securing a population of exogenous cells located within or on a surface of a cell cover, over a top of the defect 803. Creation of the hole initiates a release of precursor cells into the hole and the exogenous cells in the cell cover induce the precursor cells to form cartilage tissue. The precursor cells infiltrate the matrix material to form a cell clot or scab. An example 900 is shown in FIG. 17 whereby the matrix material 901 sits in a hole 902 that lays below the cartilage defect 903 and a cell cover 904 is secured over a top 908 of the defect 903 via a manner as shown in FIG. 12. A gap 905 exists between the cell cover 904 and the matrix material 901. Similar to the above-mentioned methods, it is believed that the exogenous cells 906 in the cell cover 904 induce the precursor cells 907 to form a more hyaline-like cartilage in the defect 903.

### EXAMPLE

Bone marrow aspirate was harvested from an allogeneic donor goat. A bone marrow stromal cell (BMSC) fraction was obtained following plastic adherence of the cells and subsequent culture expansion was performed using standard culture conditions (37° C./5% CO2) and medium (alpha-MEM/10% FCS). Cells were passaged on reaching 80% confluence (up to P3) and cryopreserved prior to use.

Treatment recipient goats, approximately 2.5 years old and 50-90 kg were used in the study. X-ray analysis confirmed normal bone mineral density. Micro fracture was performed on each goat (Group 1: N=3 micro fracture only, Group 2: N=3 micro fracture plus cell injection) as follows: A single annular defect (about 8 mm diameter) was generated in the medial femoral condyle of the stifle joint at a depth equivalent to the subchondral bone layer. A chondral pick (about 1 mm diameter) and mallet was used to perform the micro fracture procedure (about 3 mm depth, average of 7 holes per defect). After wound closer, 3 of the 6 goats received immediate injection of BMSCs into the knee joint as follows: Cells were thawed at 37° C. and washed twice with culture medium and assessed for viability using trypan blue. 1×10⁷ viable cells were mixed gently with a sodium hyaluronon (HA) solution in a syringe. Bubbles were removed from the HA/cell solution by gentle ‘flicking’ and the cell/HA solution was administered by intra articular injection. The knee was flexed and extended gently 20 times and then maintained in 70-90° flexion for 10 minutes prior to recovery. Hard casting was used to immobilise the joint for 1 week, followed by 11 weeks rehabilitation with full mobilisation.

At 12 weeks, macroscopic images were taken of each defect site before fixing in 10% neutral buffered formalin, decalcifying, and wax embedding. Tissues were sectioned (5 μm) and stained with Haematoxylin and Eosin to observe tissue infill and Safranin O to visualise the formation of glycosaminoglycan (GAG) rich new cartilage.

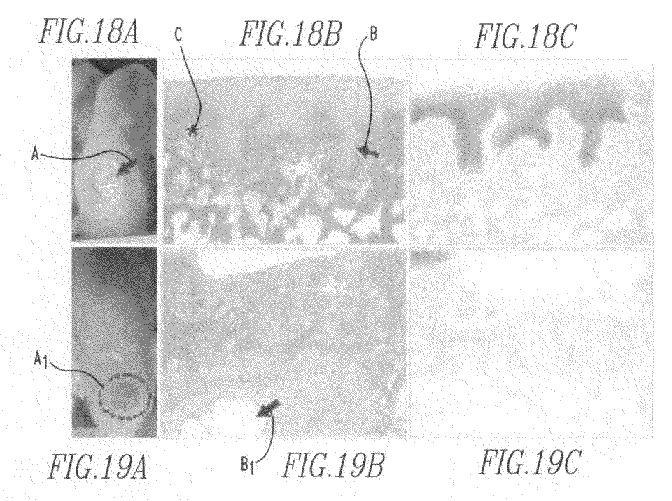

No lameness was observed in any animals. Animals that received an injection of allogeneic BMSCs did not exhibit signs of acute or chronic immune rejection. As shown in FIG. 18A, macroscopic images revealed that goats that received an injection of BMSC post microfracture showed in-fill of the defect site with white, shiny repair tissue suggestive of new cartilage A. As shown in FIG. 18B, haematoxylin and eosin staining revealed an in-fill of repair tissue in the defect and microfracture sites in goats that received an injection of BMSC post microfracture B. The repair tissue showed good integration with surrounding tissues and repair of the bone surrounding the microfracture sites was also evident C. As shown in FIG. 18C, Safranin O staining, represented by the darker color, shows the repair tissue of the goats, which received an injection of BMSC post microfracture, to be rich in GAGs and have the appearance of hyaline-like cartilage. As shown in FIG. 19A, the defects of goats that received microfracture alone showed very limited fill with new tissue and the subchondral bone remained exposed A₁. As shown in FIG. 19B, the goats that received microfracture alone show poor tissue in-fill, no repair of bone surrounding the microfracture sites, and cyst formation B₁. As shown in FIG. 19C and compared with the repair tissue shown in FIG. 18C, GAG rich repair tissue was not observed in goats receiving microfracture alone.

As various modifications could be made to the exemplary embodiments, as described above with reference to the corresponding illustrations, without departing from the scope of the disclosure, it is intended that all matter contained in the foregoing description and shown in the accompanying drawings shall be interpreted as illustrative rather than limiting. Thus, the breadth and scope of the present disclosure should not be limited by any of the above-described exemplary embodiments, but should be defined only in accordance with the following claims appended hereto and their equivalents.

What is claimed is:

1. A method of regenerating cartilage consisting of:
initiating a release of precursor cells into a cartilage defect; and
applying a population of exogenous cells to the cartilage defect,
wherein the exogenous cells induce the precursor cells to form cartilage tissue.

2. The method of claim 1 wherein initiating the release of precursor cells into a cartilage defect comprises preparing a subchondral bone surface for the release of the precursor cells.

3. The method of claim 1 wherein the exogenous cells are associated with a natural or bioabsorbable synthetic material.

4. The method of claim 3 wherein the exogenous cells are located within or on a surface of the natural or bioabsorbable synthetic material.

5. The method of claim 3 further consisting of placing a matrix material adjacent to the natural or bioabsorbable synthetic material.

6. The method of claim 5 wherein the matrix material comprises a bioabsorbable porous material.

7. The method of claim 3 wherein the natural or bioabsorbable synthetic material surrounds a matrix material.

8. The method of claim 1 wherein the precursor cells comprise bone marrow cells and progenitor cells.

9. The method of claim 8 wherein the progenitor cells are selected from a group consisting essentially of adipoprogenitor cells, osteoprogenitor cells, chondroprogenitor cells, hemapoeitic cells, and any combination thereof

10. The method of claim 1 wherein the exogenous cells are selected from a group consisting essentially of chondrocytes, synoviocytes, fat pad cells, mesenchymal stem cells, chondroprogenitor cells, and any combination thereof.

11. The method of claim 10 wherein the mesenchymal stem cells are selected from a group consisting essentially of differentiated, undifferentiated, allogenic, autologous, and any combination thereof.

12. The method of claim 1 wherein the cartilage defect comprises a full thickness or partial thickness defect.

13. The method of claim 1 wherein the exogenous cells induce the precursor cells to form cartilage tissue through a release of factors by the exogenous cells, the factors stimulating the precursor cells to form cartilage cells, the cartilage cells forming cartilage tissue.

14. The method of claim 13 wherein the factors are selected from a group consisting essentially of transforming growth factors, fibroblast growth factors, platelet-derived growth factors, insulin-like growth factors, epidermal growth factors, interleukins, and any combination thereof.

15. A method of regenerating cartilage consisting of:
initiating a release of precursor cells into a cartilage defect, the precursor cells forming a cell clot in the defect; and
securing a population of exogenous cells over a top of the defect,
wherein the exogenous cells induce the precursor cells in the cell clot to form cartilage tissue.

16. The method of claim 15 wherein initiating the release of precursor cells into a cartilage defect comprises preparing a subchondral bone surface for the release of the precursor cells.

17. The method of claim 15 wherein the exogenous cells are associated with a natural or bioabsorbable synthetic material.

18. The method of claim 17 wherein the exogenous cells are located within or on a surface of the natural or bioabsorbable synthetic material.

19. The method of claim 17 further consisting of placing a matrix material between the natural or bioabsorbable synthetic material and the defect.

20. The method of claim 19 wherein the matrix material comprises a bioabsorbable porous material.

21. The method of claim 15 wherein the precursor cells comprise bone marrow cells and progenitor cells.

22. The method of claim 21 wherein the progenitor cells are selected from a group consisting essentially of adipoprogenitor cells, osteoprogenitor cells, chondroprogenitor cells, hemapocitic cells, and any combination thereof.

23. The method of claim 15 wherein the exogenous cells are selected from a group consisting essentially of chondrocytes, synoviocytes, fat pad cells, mesenchymal stem cells, chondroprogenitor cells, and any combination thereof.

24. The method of claim 23 wherein the mesenchymal stem cells are selected from a group consisting essentially of differentiated, undifferentiated, allogenic, autologous, and any combination thereof.

25. The method of claim 15 wherein the cartilage defect comprises a full thickness or partial thickness defect.

26. The method of claim 15 wherein the exogenous cells induce the precursor cells in the cell clot to form cartilage tissue through a release of factors by the exogenous cells, the factors stimulating the precursor cells to form cartilage cells, the cartilage cells forming cartilage tissue.

27. The method of claim 26 wherein the factors are selected from a group consisting essentially of transforming growth factors, fibroblast growth factors, platelet-derived growth factors, insulin-like growth factors, epidermal growth factors, interleukins, and any combination thereof.

28. A method of regenerating cartilage comprising:
initiating a release of precursor cells into a cartilage defect; and
placing a tissue paste in the defect, the tissue paste including tissue selected from a group consisting essentially of bone, cartilage, synovium, fat pad, and any combination thereof,
wherein the precursor cells induce the tissue to form cartilage tissue.

29. The method of claim 28 wherein initiating the release of precursor cells into a cartilage defect comprises preparing a subchondral bone surface for the release of the precursor cells.

30. The method of claim 28 further comprising injecting exogenous cells into the defect, the exogenous cells inducing the tissue to form cartilage tissue.

31. The method of claim 28 further comprising securing a population of exogenous cells over a top of the defect, the population of exogenous cells inducing the tissue to form cartilage tissue.

32. The method of claim 31 wherein the exogenous cells are associated with a natural or bioabsorbable synthetic material.

33. The method of claim 32 wherein the exogenous cells are located within or on a surface of the natural or bioabsorbable synthetic material.

34. The method of claim 30 wherein the exogenous cells are selected from a group consisting essentially of chondrocytes, synoviocytes, fat pad cells, mesenchymal stem cells, chondroprogenitor cells, and any combination thereof.

35. The method of claim 31 wherein the exogenous cells are selected from a group consisting essentially of chondrocytes, synoviocytes, fat pad cells, mesenchymal stem cells, chondroprogenitor cells, and any combination thereof.

36. The method as in claim 34 or 35 wherein the mesenchymal stem cells are selected from a group consisting essentially of differentiated, undifferentiated, allogenic, autologous, and any combination thereof.

37. The method of claim 28 wherein the cartilage defect comprises a full thickness or partial thickness defect.

38. The method in claim 28, 30, or 31 wherein the precursor cells or exogenous cells induce the tissue to form cartilage tissue through a release of factors by the precursor or exogenous cells, the factors stimulating the tissue to form cartilage tissue.

39. A method of regenerating cartilage consisting of:
initiating a release of precursor cells into a cartilage defect, the precursor cells forming a cell clot in the defect; and
injecting the cartilage defect with exogenous cells,
wherein the exogenous cells induce the precursor cells in the cell clot to form cartilage tissue.

40. The method of claim 39 wherein initiating the release of precursor cells into a cartilage defect comprises preparing a subchondral bone surface for the release of the precursor cells.

41. The method of claim 39 further consisting of placing a matrix material between the exogenous cells and the defect.

42. The method of claim 41 wherein the matrix material comprises a bioabsorbable porous material.

43. The method of claim 39 wherein the precursor cells comprise bone marrow cells and progenitor cells.

44. The method of claim 43 wherein the progenitor cells are selected from a group consisting essentially of adipoprogenitor cells, osteoprogenitor cells, chondroprogenitor cells, hemapoeitic cells, and any combination thereof.

45. The method of claim 39 wherein the exogenous cells are selected from a group consisting essentially of chondrocytes, synoviocytes, fat pad cells, mesenchymal stem cells, chondroprogenitor cells, and any combination thereof.

46. The method of claim 45 wherein the mesencbymal stem cells are selected from a group consisting essentially of differentiated, undifferentiated, allogenic, autologous cells, and any combination thereof.

47. The method of claim 39 wherein the cartilage defect comprises a full thickness or partial thickness defect.

48. The method of claim 39 wherein the exogenous cells induce the precursor cells in the cell clot to form cartilage tissue through a release of factors by the exogenous cells, the factors stimulating the precursor cells to form cartilage cells, the cartilage cells forming cartilage tissue.

49. The method of claim 48 wherein the factors are selected from a group consisting essentially of transforming growth factors, fibroblast growth factors, platelet-derived growth factors, insulin-like growth factors, epidermal growth factors, interleukins, and any combination thereof.

50. A method of regenerating cartilage comprising:
creating at least one hole in a bone lying below a cartilage defect, wherein creating the at least one hole initiates a release of precursor cells into the hole;
placing a matrix material into the at least one hole; and
securing a population of exogenous cells over a top of the defect, wherein the exogenous cells induce the precursor cells to form cartilage tissue.

51. The method of claim 50 wherein the precursor cells infiltrate the matrix material to form a cell clot.

52. The method of claim 50 wherein the exogenous cells are associated with a natural or bioabsorbable synthetic material.

53. The method of claim 52 wherein the exogenous cells are located within or on a surface of the natural or bioabsorbable synthetic material.

54. The method of claim 50 wherein the matrix material comprises a bioabsorbable porous material.

55. The method of claim 50 wherein the precursor cells comprise bone marrow cells and progenitor cells.

56. The method of claim 55 wherein the progenitor cells are selected from a group consisting essentially of adipoprogenitor cells, osteoprogenitor cells, chondroprogenitor cells, hemapoeitic cells, and any combination thereof.

57. The method of claim 50 wherein the exogenous cells are selected from a group consisting essentially of chondrocytes, synoviocytes, fat pad cells, chondroprogenitor cells, mesenchymal stem cells, and any combination thereof.

58. The method of claim 57 wherein the mesenchymal stem cells are selected from a group consisting essentially of differentiated, undifferentiated, allogenic, autologous, and any combination thereof.

59. The method of claim 50 wherein the cartilage defect comprises a full thickness or partial thickness defect.

60. The method of claim 50 wherein the exogenous cells induce the precursor cells to form cartilage tissue through a release of factors by the exogenous cells, the factors stimulating the precursor cells to form cartilage cells, the cartilage cells forming cartilage tissue.

61. The method of claim 60 wherein the factors are selected from a group consisting essentially of transforming growth factors, fibroblast growth factors, platelet-derived growth factors, insulin-like growth factors, epidermal growth factors, interleukins, and any combination thereof.

62. The method of claim 28 wherein the tissue is in a bioabsorbable carrier.

63. The method of claim 30 wherein the exogenous cells are in a bioabsorbable carrier.

64. The method of claim 39 wherein the exogenous cells are in a bioabsorbable carrier.
